(19) United States
(12) Patent Application Publication
(10) Pub. No.: US 2015/0174418 A1
(43) Pub. Date: Jun. 25, 2015

(54) Device and Methods for Noninvasive Neuromodulation Using Targeted Transcranial Electrical Stimulation

(71) Applicant: THYNC, INC., Los Gatos, CA (US)

(72) Inventors: William J. TYLER, Newton, MA (US); Daniel Z. WETMORE, San Francisco, CA (US); Alexander OPITZ, Goettingen (DE); Tomokazu SATO, Pasadena, CA (US); Sumon PAL, Boston, MA (US)

(21) Appl. No.: 14/576,588

(22) Filed: Dec. 19, 2014

Related U.S. Application Data
(63) Continuation of application No. PCT/US13/47174, filed on Jun. 21, 2013.
(60) Provisional application No. 61/663,409, filed on Jun. 22, 2012.

(51) Int. Cl. A61N 1/372; G06F 19/00; G06T 17/20; A61B 19/00; A61N 7/00

(52) U.S. Cl. CPC A61N 1/37264; A61B 19/50; A61N 7/00; G06T 17/20; G06F 19/3437

(57) ABSTRACT

A system for transcranial electrical stimulation comprises a processor having instructions of a computer model that can be adjusted to the head subject in response to one or more input parameters. The adjustable model may comprise a plurality of structures that can be adjusted to the head of the subject in response to parameters that can be readily measured, such as head size and head shape. Discrete brain regions can be stimulated with a plurality of electrodes arranged to stimulate the targeted region with decrease stimulation of the non-targeted regions in order to improve subject comfort. In many embodiments the plurality of electrodes comprises a montage of electrodes, and the targeted location is identified on the adjustable model and the number of electrodes, locations and pulse parameters determined in response to the adjustable model. The adjustment can be helpful to align structures of model with structures of the subject.

### CROSS-REFERENCE TO RELATED APPLICATIONS

This application is a continuation application of PCT/US2013/047174 filed on Jun. 21, 2013, which claims the benefit of priority of U.S. Provisional Patent Application No. 61/663,409 filed Jun. 22, 2012, the entire disclosures of which are incorporated herein by reference.

### 1. FIELD OF THE INVENTION

The present invention relates to methods and systems for noninvasive neuromodulation using targeted transcranial electrical stimulation (hereinafter “TES”).

The brain is composed of neurons and other cell types in connected networks that process sensory input, generate motor commands, and control all other behavioral and cognitive functions. Neurons communicate primarily through electrochemical pulses that transmit signals between connected cells within and between brain areas. Stimulation technologies that affect electric fields and electrochemical signaling in neurons can modulate the pattern of neural activity and cause altered behavior, cognitive states, perception, motor output, and more.

One challenge for neuromodulation is targeting the appropriate area to stimulate, inhibit, or modulate. In man, invasive preparations using implanted deep brain stimulation electrodes are used for therapeutic intervention in epilepsy, Parkinson's disease, and other disorders when drugs or other less invasive strategies are ineffective. Due to the cost, pain, and risk of invasive brain surgery, noninvasive techniques for neuromodulation are preferable.

Technologies for transmitting focused energy into the skull to modify brain activity include transcranial magnetic stimulation (hereinafter “TMS”), transcranial ultrasound neuromodulation, and electrical stimulation.

Although prior methods and apparatus have been proposed to treat the brain through the scalp, these prior methods and apparatus can produce less than ideal results in at least some instances. For example, transcranial electrical stimulation can result in side effects such as a tingling, scratching or itching sensation in at least some instances. Further, the amount and distribution of current of prior transcranial electrical stimulation can be less than ideal and provide less than ideal therapeutic results in at least some instances. Electrical stimulation through scalp electrodes has been used to affect brain function in man using both transcranial alternating current stimulation (hereinafter “tACS”) and transcranial direct current stimulation (hereinafter “tDCS”), two forms of transcranial electric stimulation (TES). Although relative to tDCS, tACS offers the advantage of reductions in pain, tingling, and other side effects on the scalp, these side effects can still be present in at least some instances. Another strategy to reduce these side effects is to use a high-density-tDCS (hereinafter “HD-tDCS”) system with smaller electrode pads, such as the one sold by Soterix Medical. However, such systems can be overly complex, and may provide less than ideal results in at least some instances.

Although TES has been proposed for modulating brain activity and cognitive function in man, the prior systems and methods have produced less than ideal results in at least some instances. Prior systems and methods for TES have been disclosed (see for example, Capel U.S. Pat. No. 4,646,744; Haimovich et al. U.S. Pat. No. 5,540,736; Besio et al. U.S. Pat. No. 8,190,248; Hagedorn and Thompson U.S. Pat. No. 8,239,030; Bikson et al. US patent application 2011/0144716; and Lebedev et al. US patent application 2009/0177243). Other systems described in the prior art require surgical implantation of components for electrical stimulation on the head of a user (see for example Gliner U.S. Pat. Nos. 8,121,695 and 8,433,414). tDCS systems with numerous electrodes and a high level of configurability have been disclosed (see for example Bikson et al. US Patent applications 2012/0209346, 2012/0265261, and 2012/0245653), as have portable TES systems for auto-stimulation (Brocke US Patent application 2011/0288610 and Tanaka and Nakanishi U.S. Pat. No. 8,150,537).

It would be helpful to have effective strategies for targeting electric fields to activate one or more regions of interest without activating areas outside the region(s) of for achieving effective neuromodulation with TES. However, the prior strategies for targeting deep brain regions with electric fields can be less than ideal in at least some instances. For example, the electrodes can be less specific than would be ideal and stimulate tissue away from the target area. In at least some embodiments, the size of the electrodes can be oversized in order to decrease side effects such as itching and tingling sensations. However, the increased size of these electrodes can result in stimulation of a greater area of the brain than would be ideal. Variability among tissues and individuals may contribute to the less than ideal results of the prior methods and apparatus.

Different tissue types and brain regions exhibit distinct conductivity, and the orientation of these tissues introduces an inhomogeneous and anisotropic conductivity that distort the electric field in a non-trivial way. For instance, injected current can be shunted through the scalp or diffuse through highly conductive cerebro-spinal fluid. The prior art methods and apparatus attempting to address variable conductivity can be less than ideal in at least some instances.

Although finite element models (hereinafter “FEMs”) have been proposed to estimate underlying current sources and sinks in the brain based on the structure and electrical properties of brain and head anatomy, such prior FEM modeling can be less than ideal in at least some instances. For example, FEM is computationally expensive and complex. At least some of the prior approaches have relied on patient tomography, which can be more complex than would be ideal such that at least some patients may not receive therapies in at least some instances. U.S. patent application Ser. No. 13/294,994 by inventors Bikson et al. describes estimating current flow in tissue using an FEM.

Transcranial ultrasound neuromodulation employs ultrasound for stimulating neural tissue. Patent applications have described the use of transcranial ultrasound to activate, inhibit, or modulate neuronal activity, for example U.S. patent application Ser. No. 13/003,853 and PCT patent application PCT/US2010/055527.

TMS induces electric fields in the brain by generating a strong, generally pulsed, magnetic field with a coiled electromagnet or other magnetic field at or near the head. TMS has been used for research and therapies such as for intractable depression.

Deep brain stimulation (hereinafter “DBS”) electrodes deliver current to a targeted brain area near implanted electrodes. Although DBS can be an effective treatment for treating Parkinson's disease in patients unresponsive to drugs, the implantation of electrodes can be more invasive than would be ideal.

Optogenetic stimulation uses light of a specified wavelength to activate an engineered protein expressed in neurons or other cell types that modifies the electrical and/or biochemical activity of a targeted cell. For deep brain applications, light is generally introduced via an implanted optical fiber, which can be invasive. Also, the use of an engineered protein can limit the usefulness of this approach.

Electrocorticography (hereinafter “ECoG”) arrays are electrodes implanted on the surface of the brain or dura. Although ECoG arrays can record electrical potentials and/or stimulate underlying cortical tissue, for instance to map the focal point of a seizure, the implantation of arrays can be more invasive than would be ideal.

Affecting brain function with TES protocols is an active field of research. However, the prior methods and apparatus produce less than ideal results in at least some instances. In light of the above, it would be helpful to provide improved systems and methods to treat patients with TES. Ideally, such systems and methods would improve the convenience, consistency, targeting, comfort, automation, effectiveness, and/or safety of TES for patients, health care professionals and other individuals. Also, improved systems and methods would deliver electrical stimulation to a targeted brain region while reducing current density in non-targeted brain regions for achieving specific forms of neuromodulation with decreased and ideally minimal side effects. Various embodiments as described herein overcome at least some of the above deficiencies of the prior methods and systems.

### SUMMARY

Embodiments of the present invention provide improved systems and methods for targeted transcranial electrical stimulation (TES) to induce neuromodulation and overcome at least some of the deficiencies of the prior systems and methods. In many embodiments a processor comprises instructions of an adjustable model that can be adjusted to the head and brain anatomy of a subject in response to one or more input parameters. The adjustable model may comprise a plurality of structures that can be adjusted to the head of the subject in response to parameters that can be readily measured, such as head size and head shape. The input parameters may comprise parameters of one or more clinically identifiable indicia of head size and shape such as distances between prominent points, which may comprise one or more of a nasion-inion distance, a left ear-right ear distance, or distance from a central location. Discrete brain regions can be stimulated with a plurality of electrodes arranged to stimulate the targeted region with decrease stimulation of the non-targeted regions in order to improve subject comfort. In many embodiments, the number of electrodes, locations, and pulse parameters of the electrodes are determined in response to the adjustable model in order to decrease peak current of electrodes. In many embodiments the plurality of electrodes comprises a montage of electrodes, and the targeted location is identified on the adjustable model and the number of electrodes, locations and pulse parameters determined in response to the adjustable model. The adjustment can be helpful to align structures of the model with structures of the subject. In many embodiments, the plurality of structures of the model corresponds to one or more tissue structures comprising one or more of grey matter, white matter, skull, cerebrospinal fluid (CSF), scalp, muscle, air, electrode or gel, and a location of each of the plurality of structures is adjusted based on the input data.

The adjustable model may comprise a finite element model or finite difference model adjusted based on the one or more input parameters, such that the positions of the electrodes can be determined without tomographic imaging of the subject. In many embodiments, the finite element model comprises a mesh composed of a plurality of finite elements, and locations of each of the plurality of finite elements can be adjusted in order to position each of the plurality of elements at locations corresponding to structures of the subject. In many embodiments, a plurality of finite elements is provided for a plurality of tissues of the subject, and locations of the finite elements are adjusted in response to the subject data.

Computational models are advantageous for modeling the transmission of electric fields in the brain based on realistic head and brain anatomy to determine the number and location of electrodes and stimulation parameters. Finite element models can be used to model electric fields in the brain and can be used to determine electrode montages and stimulation parameters for targeted TES, in accordance with at least some embodiments. One or more appropriate brain regions can be selected to achieve a specified neuromodulatory effect, then TES is targeted to these brain regions based on a FEM or other computational model that estimates electric fields in the brain. In an embodiment, a system comprises a processor configured to compute a FEM model to estimate current densities in the brain due to stimulation from two or more TES electrodes.

In many embodiments, neuromodulation is targeted to more than one brain region. In these embodiments, targeted TES or another technique for neuromodulation target a first brain region to induce a set of behavioral, cognitive, or other effects, while concurrently (or in close temporal relation) targeting a second brain regions to counteract a subset of the effects of stimulation targeting the first brain region. In this manner, the functional effect of neuromodulation can be shaped to reduce unwanted side effects.

In many embodiments, the timing of targeted TES is designed to modulate brain activity that occurs in the temporal domain, including brain rhythms and spatiotemporal patterns of neural activity between connected brain circuits.

In many embodiments, brain recordings and/or physiological monitoring are used to measure the effect of targeted TES. This technique is advantageous for providing feedback (in some embodiments, real-time feedback) concerning the targeting, timing, and stimulation parameters for targeted TES and/or other techniques for neuromodulation used.

In many embodiments, a device assists a user in placing electrodes at appropriate locations to achieve a desired form of neuromodulation.

In many embodiments, TES electrodes comprise one or more of being positionable on portions of the head that do not have hair; having a semi-permeable sack between the electrode and the skin that releases a small amount of water or other electrically conductive material when squeezed; or being incorporated in an array of electrodes in a single high-density assembly. In many embodiments, a system comprises a processor configured to compute a FEM model that selects electrode locations to be on regions of the head, face, neck, or other body area that do not have hair.

In many embodiments, the system is portable and battery powered.

In many embodiments, targeting is personalized based on structural imaging of a user's head and brain.

In many embodiments, the placement of TES electrodes and spatiotemporal pattern of stimulation delivered through the TES electrodes is configured for targeting the ventromedial prefrontal cortex (hereinafter “VmPFC” and also referred to as VmPFC Brodmann area 10). Targeting to the VmPFC can be advantageous for modulating emotion, risk, decision-making, and fear.

In many embodiments, the placement of TES electrodes and spatiotemporal pattern of stimulation delivered through the TES electrodes is configured for targeting the orbitofrontal cortex (hereinafter “OFC” and also referred to as OFC Brodmann areas 10, 11, 14). Targeting to the OFC can be advantageous for modulating executive control and decision making.

In many embodiments, the placement of TES electrodes and spatiotemporal pattern of stimulation delivered through the TES electrodes is configured for targeting the ventral striatum. Targeting to the ventral striatum can be advantageous for modulating emotional and motivational aspects of behavior.

In many embodiments, the placement of TES electrodes and spatiotemporal pattern of stimulation delivered through the TES electrodes is configured for targeting the locus coeruleus (LC). Targeting to the LC can be advantageous for modulating norepinephrinergic tone, learning and memory, sleep, processing of stressful stimuli, and other effects.

In many embodiments, the placement of TES electrodes and spatiotemporal pattern of stimulation delivered through the TES electrodes is configured for targeting the ventral tegmental area (hereinafter VTA). Targeting to the VTA can be advantageous for modulating reward circuitry, motivation, drug addiction, intense emotions relating to love, and other effects mediated by this dopaminergic system.

In many embodiments, systems and methods provide TES targeting based on an FEM or other suitable computational model. In many embodiments, an adjustable model of the head captures common anatomical features to make the anatomical model more accurate for a particular individual. The adjustable model of the head may comprise standard model of the head, based on normal anatomical and physiological values of a population. The computationally intensive steps of generating the standard model that is input to the FEM or other suitable computational model can be pre-computed and provide with the system prior to input of subject specific parameters, such that determination of the electrode location and parameters provide computationally simpler adjustments for an individual. In many embodiments, a processor is configured to adjust a standard model and use the adjusted standard model in a FEM or other suitable computational model for determining estimates of current density and direction for a particular individual subject.

Higher peak currents can be uncomfortable in TES, and in many embodiments systems and methods are provided for reducing peak currents delivered while stimulating targeted brain regions at similar current densities. In many embodiments, a FEM method determines a TES stimulation protocol and electrode montage that increases the number of electrodes, reduces the peak current delivered, and still targets the same brain region with a similar current density. In many embodiments, system comprising a processor configured to perform FEM and determine based on the FEM results a TES stimulation protocol and electrode montage that increases the number of electrodes, reduces the peak current delivered, and still targets the same brain region with a similar current density.

Many embodiments advantageously combine TES with another neuromodulation technique for creating patterns of brain stimulation. The FEM can be used to estimate, optimize, and/or improve targeting of induced currents in the brain due to combined stimulation with TES and another technique for neuromodulation comprising a processor configured for using FEM to estimate, optimize, and/or improve targeting of induced currents in the brain due to combined stimulation with TES and another technique for neuromodulation.

In at least some embodiments constructive and destructive interference between currents delivered from three or more sets of electrodes affects the density and direction of currents induced in the brain. FEM can be used to improve targeting of induced currents in the brain delivered by three or more electrodes operating in pulsed-mode operation and using phase-shifting of the pulses relative to each other by improving or optimizing electrode configuration and electrostimulation parameters. In many embodiments, the system comprises a processor configured to use FEM to estimate, optimize, and/or improve targeting of induced currents in the brain from three or more electrodes operating in pulsed-mode operation and using phase-shifting of the pulses relative to each other to target one or more brain region.

In a first aspect, embodiments provide an apparatus for use with a brain of a subject. The apparatus comprises an input to receive data of the subject. A computer is configured with an adjustable model to determine parameters and electrode positions to provide a spatiotemporal pattern of stimulation in order to target one or more regions of the brain with electrical stimulation based on the input.

In many embodiments, the adjustable model comprises a plurality of structures corresponding to tomography of another subject, and wherein the model is adjusted based on the input in order to align the structures of the model with corresponding structures of the subject. Each of the plurality of structures may correspond to one or more of grey matter, white matter, skull, cerebrospinal fluid (CSF), scalp, muscle, air, electrode, or gel and wherein a location of each of the plurality of structures is adjusted based on the input.

In many embodiments, the adjustable model is scaled to the subject in response to the input in order to align structures of the model with structures of the subject.

In many embodiments, the adjustable model comprises a finite element model comprising a mesh composed of a plurality of finite elements, and the mesh and the plurality of finite elements are scaled to the subject based on the input. The computer can be configured to decrease a peak current in order to stimulate a target region of the brain based on the input.

In many embodiments, computer is configured with the adjustable model to estimate a current induced in the brain by transcranial electrical stimulation treatment of the subject, and the computer comprises,

the adjustable model, the adjustable model based at least in part on brain and head anatomy of another subject based on a structural scan of the another subject and stored in a computer readable memory of the computer,

a database or lookup table indicating adjustments to the adjustable model of brain and head anatomy based on at least one adjustment parameter, and

a processor configured to load the adjustable model of brain and head anatomy from the computer readable memory, to determine one or more model adjustments to make in response to querying a database or lookup table, and to compute adjustment to the adjustable model of brain and head anatomy.

In many embodiments, the processor is configured to compute a computational model for estimating current density and direction in the brain based on the input and the adjustable model.

In many embodiments, the processor comprises instructions to determine one or more model adjustment parameters, the one or more model adjustment parameters comprising a subject measurement comprising of one or more of:
a subject's skull, a scalp, a hair, a face, a head, a dura, a brain, a neck, or other part of the body;
a cognitive assessment comprising one or more of a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task;
a physiological measurement of the body comprising of one or more of electromyogram (EMG), galvanic skin response (GSR), heart rate, blood pressure, respiration rate, electrocardiogram (EKG), pulse oximetry (e.g. photoplethysmography), heart rate, pupil dilation, eye movement, or gaze direction;
a subject metadatum comprising one or more of gender, height, weight, age, diet, pharmaceutical drugs used, cognitive abilities, cognitive disabilities, or other metadata; or
a subject genetic datum including one or more of microduplication, microdeletion, single nucleotide polymorphism (SNP), aneuploidy, allele, or other genetic data.

In many embodiments, the processor is configured to write the adjusted model of brain and head anatomy to a computer readable memory and determine positions of the electrodes in order to decrease peak current.

In many embodiments, the apparatus further comprises a communication system for transmitting information between a remote processor and a transcranial electrical stimulation system controller. In many embodiments, the communication system comprises the Internet.

In many embodiments, the transmitted information comprise a Model Adjustment Parameter transmitted from a transcranial electrical stimulation system controller to a remote server.

In many embodiments, the transmitted information comprises a transcranial electrical stimulation electrode montage transmitted to a transcranial electrical stimulation system controller.

In many embodiments, the transmitted information comprises a transcranial electrical stimulation electrostimulation protocol transmitted to a TES system controller.

In many embodiments, the transmitted information comprises a transcranial electrical stimulation system parameters selected from the group consisting of: firmware version, number of electrodes, location of electrodes, size and shape of electrodes, stimulation protocol history, capacity of the system to deliver direct current stimulation and/or alternating current stimulation, battery charge remaining, maximum current deliverable, constraints on anode-cathode pairs that can be created from available electrodes, and other information about a TES system.

In many embodiments, the transmitted information comprises at least one brain target for transcranial electrical stimulation.

In many embodiments, the transmitted information relates to the outcome of one or more previous TES sessions, where the transmitted data comprises one or more of a subjective assessment by the subject or another individual, a cognitive assessment, a brain recording or other physiological measurement, or other outcome assessment.

In many embodiments, the transmitted information comprises instructions to a TES system controller to adjust one or more parameters comprising one or more of an electrode position, anode-cathode pairing of two or more electrodes, current delivered from an anode-cathode pair of electrodes, timing of stimulation from electrodes, or frequency of alternating current stimulation, or other TES parameter.

In another aspect, embodiments provide an apparatus for determining a transcranial electrical stimulation electrode montage and electrostimulation protocol. The apparatus comprises a processor configured to compute an FEM model based on a set of two or more electrodes and determine additional electrode positions and stimulation parameters.

In many embodiments, the processor comprises instructions such that a high current delivered from an anode-cathode pair is replaced by a larger number of electrodes delivering a lower current than the high current while approximately maintaining the induced current in one or more brain regions.

In many embodiments, the apparatus further comprises components to deliver another brain stimulation by one or more of transcranial ultrasound neuromodulation, transcranial magnetic stimulation (TMS), deep brain stimulation (DBS), optogenetic stimulation, one electrode or an array of electrodes implanted on the surface of the brain or dura (electrocorticography (ECoG) arrays), or radio-frequency stimulation. The components to deliver another brain stimulation can be triggered with a pre-defined temporal relationship relative to a TES protocol. In many embodiments, the components to deliver another brain stimulation is delivered concurrently with a TES protocol.

In many embodiments, the apparatus further comprises circuitry and multiple electrode pairs configured to be pulsed at defined latencies relative to each other to target electrical stimulation to one or more brain regions. The circuitry and multiple electrodes may be configured for pulses comprising one or more of direct current stimulation, alternating current stimulation, or both direct current stimulation or alternating current stimulation. The circuitry and multiple electrodes can be configured for pulsed electrical stimulation from with a phase shift between pulses from different sets of electrodes of less than 10 milliseconds (hereinafter “ms”).

In another aspect, embodiments provide a method of treating a patient, the method comprises receiving input data of the subject, and adjusting a model to determine parameters and electrode positions to provide a spatiotemporal pattern of stimulation in order to target one or more regions of the brain with electrical stimulation based on the input.

### BRIEF DESCRIPTION OF THE DRAWINGS

The embodiments of the present disclosure have other advantages and features which will be more readily apparent from the following detailed description of the invention and the appended claims, when taken in conjunction with the accompanying drawings, in which:

FIG. 1 shows a schematic of pulsed transcranial alternating current stimulation (tACS), in accordance with embodiments;

FIG. 2 shows a workflow for adjusting targeting of TES, in accordance with embodiments;

FIG. 3a shows example tACS waveforms, in accordance with embodiments;

FIG. 3b shows a schematic representation of pulsed TES delivered from three electrodes, in accordance with embodiments;

FIG. 4 shows different views of a computerized representation of a standard model head with eight electrodes and modeled targeting of electric fields, in accordance with embodiments;

FIG. 5a shows the relative current delivered from eight electrodes for targeting TES to right prefrontal cortex, in accordance with embodiments;

FIG. 5b shows the current direction in the brain estimated by an FEM simulation of eight electrodes for targeting TES to right prefrontal cortex, in accordance with embodiments;

FIG. 5c shows the current intensity in the brain estimated by an FEM simulation of eight electrodes for targeting TES to right prefrontal cortex, in accordance with embodiments;

FIG. 6a shows the relative current delivered from eight electrodes for targeting TES to left prefrontal cortex, in accordance with embodiments;

FIG. 6b shows the current direction in the brain estimated by an FEM simulation of eight electrodes for targeting TES to left prefrontal cortex, in accordance with embodiments;

FIG. 6c shows the current intensity in the brain estimated by an FEM simulation of eight electrodes for targeting TES to left prefrontal cortex, in accordance with embodiments;

FIG. 7 shows a schematic representation of the brain and a target location for TES at orbitofrontal cortex, in accordance with embodiments;

FIG. 8 shows a method of generating a volume mesh for calculating the electric field induced in the brain, in accordance with embodiments; and

FIG. 9 shows an apparatus to treat a patient in accordance with many embodiments.

### DETAILED DESCRIPTION

Transcranial electrical stimulation (TES) is advantageous for modulating brain activity and cognitive function in subjects such as human subjects. The embodiments described herein can be combined in one or more of many ways to provide advantageous devices, systems, and methods for targeted transcranial electrical stimulation (TES). Neurons and other cells in the brain are electrically active, so stimulation using electric fields is an effective strategy for modulating brain function. In various embodiments, the effect of neuromodulation induced by TES is one or more of inhibition, excitation, or modulation of neuronal activity.

The embodiments as disclosed herein can be combined with one or more of many known therapies and therapeutic devices. The embodiments as disclosed herein can be combined with each other, provided such combination is consistent with the disclosed embodiments.

As used herein a workflow encompasses a method.

As used herein, a subject encompasses an animal that can be treated with the system, and the animal can be human.

As used herein a user encompasses a person who uses the system, who may be a subject treated with the system or a medical professional.

Systems, devices, and methods for TES are described which are beneficial for generating targeted noninvasive TES for neuromodulation affecting brain regions that mediate sensory experience, motor performance, and the formation of ideas and thoughts, as well as states of emotion, physiological arousal, sexual arousal, attention, creativity, relaxation, empathy, connectedness, and other cognitive states.

Systems, devices, and methods for transcranial electrical stimulation (TES) are disclosed which are beneficial for generating targeted noninvasive TES for neuromodulation affecting brain regions that mediate sensory experience, motor performance, and the formation of ideas and thoughts, as well as states of emotion, physiological arousal, sexual arousal, attention, creativity, relaxation, empathy, connectedness, and other cognitive states. TES includes transcranial direct current stimulation (tDCS), transcranial alternating current stimulation (tACS), transcranial random noise stimulation, and pulsed transcranial electrical stimulation. Computational models for estimating electric fields generated by patterned electrical stimulation through multiple electrodes such as finite element models (FEMs) are used to determine electrode montages and stimulus protocols for generating restricted electric fields at any location specified in the brain. Targeting multiple brain regions is an effective strategy for achieving cognitive or behavioral effects in domains mediated by integrated circuits of brain regions. Configurations that combine TES with other brain stimulation modalities such as transcranial magnetic stimulation and transcranial ultrasound neuromodulation permit further specificity of induced brain changes. Configurations that include systems for recording brain activity and/or monitoring physiological markers is an effective strategy for improving the targeting of brain stimulation to achieve a desired neurocognitive effect. Improved targeting can be achieved by personalized modeling of electric fields based on anatomical imaging of the head, including the skull and brain.

TES can include stimulation using both transcranial direct current stimulation (tDCS) and transcranial alternating current stimulation (tACS). Unlike other forms of energy that can be transmitted transcranially such as ultrasound, transmission of an electrical field in the brain occurs at the speed of light and can be instantaneous on biological timescales. TES stimulation can be delivered by electrodes placed on and electrically coupled to the scalp. TES electrodes can vary in material, size, shape, number, and density in different TES stimulation paradigms in manners known to those skilled in the art. The selection of these electrode montage parameters, as well as the stimulation protocol delivered through each electrode, determines the spatiotemporal profile of the induced electric field in the brain and, accordingly, the induced effect on cognitive function, behavior, sensory processing, motor output, emotion, arousal, etc.

Steps for targeted TES may include one or more of the following: (1) Select one or more brain regions to target to achieve a desired change in brain activity; (2) Generate a sufficiently realistic anatomical model of a user's head and brain; (3) Compute a finite element model (FEM) of the electric field for a specific anatomy and electrode configuration; (4) Use the pre-computed FEM to optimize the electrode stimulation parameters required to achieve a spatiotemporal pattern of targeted TES; (5) Place the electrodes in the appropriate locations on the subject's head (which may be done by the user); and (6) Deliver electrical stimuli with the appropriate timing, duration, frequency, and intensity through each electrode specified by the computational model for targeted TES.

Hardware and software systems for TES may comprise one or more of the following components: a battery or power supply safely isolated from mains power; control hardware and/or software for triggering a TES event and controlling the waveform, duration, intensity, and other parameters of stimulation of each electrode; and one or more pairs of electrodes with gel, saline, or another material for electrical coupling to the scalp. Advantageous components of the present invention also include a preceding step of computational modeling to determine parameters and electrode positions to achieve a desired spatiotemporal pattern of stimulation. The electrode pads can be sized in one or more of many ways.

Other skin surface mounted electrodes known to one skilled in the art can be employed in TES, in accordance with embodiments described herein. In some embodiments, stimulation electrodes are adhesive and maintain positioning by adhering to the scalp. In other embodiments, a band, helmet, or other head-mounted assembly maintains the positioning of the stimulation electrodes.

Commercially available systems for providing a specified stimulus waveform to one or more pairs of TES electrodes are available from Digitimer Ltd., Welwyn Garden City, Hertfordshire, U.K. Such systems are suitable for incorporation in accordance with embodiments described herein.

The embodiments may comprise one or more components of commercial systems for generating protocols for alternating and/or direct current stimulation defined by timing relative to an external signal, timing relative to a real-time clock (or alarm), phase relationships between electrode pairs, amplitude modulation, and pulsing are known in the art of electrical engineering, digital signal processing, and analog circuit design are suitable for combination in accordance with embodiments described herein.

Although many forms of electrical stimulus can be used such as one or more of pulsed, alternating current (hereinafter “AC”), or direct current (hereinafter “DC”), in many embodiments the current comprises AC current. AC can permit higher current intensities for AC with decreased discomfort, for example without discomfort. The electrodes can be sized and placed to inhibit discomfort of the subject in accordance with embodiments described herein. In many embodiments, the current delivered through a single electrode is chosen from the group including but not limited to: less than about 10 mA, less than about 5 mA, less than about 4 mA, less than about 3 mA, less than about 2 mA, less than about 1 mA, less than about 0.5 mA, less than about 0.25 mA, less than about 0.1 mA, less than about 50 μA, less than about 25 μA, less than about 10 μA, less than about 5 μA, less than about 1 μA, less than about 0.5 μA, and less than about 0.1 μA. In advantageous embodiments, the sum of currents transmitted by all or a subset of electrodes is limited to a maximum instantaneous level chosen from the group including but not limited to: less than about 10 mA, less than about 5 mA, less than about 4 mA, less than about 3 mA, less than about 2 mA, less than about 1 mA, less than about 0.5 mA, less than about 0.25 mA, less than about 0.1 mA, less than about 50 μA, less than about 25 μA, less than about 10 μA, less than about 5 μA, less than about 1 μA, less than about 0.5 μA, and less than about 0.1 μA. In some embodiments, the maximum current level permitted for a single electrode or group including but not limited to electrodes is an average or cumulative value over a period of time chosen from the group including but not limited to: less than about 100 minutes; less than about 30 minutes; less than about 10 minutes; less than about 5 minutes; less than about 2 minutes; less than about 1 minute; less than about 30 seconds; less than about 10 seconds; less than about 5 seconds; less than about 2 seconds; less than about 1 seconds; less than about 300 milliseconds less than about 100 milliseconds; less than about 50 milliseconds; less than about 10 milliseconds; less than about 5 milliseconds; or less than about 1 millisecond.

In many embodiments that use tACS, the device is configured to deliver alternating current at one or more frequencies between about 0.01 Hz and about 500 Hz. Particularly advantageous frequencies for tACS are at frequencies of brain rhythms that naturally occur between about 0.5 Hz and about 130 Hz. In some embodiments, the components of the system that deliver alternating current stimulation are configured to deliver time-varying patterns of electrical stimulation with one or more dominant frequencies at a biologically relevant range of between about 0.01 Hz and about 500 Hz.

In some embodiments, modulation of neural activity affects the amplitude or phase of brain rhythms.

In some embodiments, the device is configured so that the modulation of brain rhythms is localized to a specific brain region.

In some embodiments, the device is configured so that the modulation of brain rhythms affects brain rhythms that occur between multiple brain regions. In some cases, functionally connected brain regions are anatomically nearby, while in other cases they are distant. The communication between related brain regions underlies important forms of cognitive function, so modulating brain rhythms between brain regions is advantageous for affecting cognitive processes.

The number and placement of TES electrodes, as well as the stimulation parameters for each electrode determine the effect induced in the brain. In some embodiments, the device is configured so that the neuromodulation induced by TES is mediated at least in part by neurons. In alternative embodiments, the device is configured so that the neuromodulation induced by TES is mediated at least in part by non-neuronal cells. In some embodiments, the device is configured so that the induced electric field has higher intensity in one or more targeted white matter tracts. In alternative embodiments, the device is configured so that the induced electric field has higher intensity in one or more targeted regions of grey matter. In some embodiments, the directionality of one or more electrical fields is modulated during a TES session. In alternative embodiments, the location and/or intensity of one or more electrical fields is modulated during a TES session.

In some embodiments, one or more dominant frequencies of tACS stimulation is individualized for a user based on their own endogenous brain rhythms. The peak frequency for behaviorally relevant rhythms such as alpha rhythms can vary by several Hz between individuals. Thus, in some embodiments, the device is configured to modulate alpha or other rhythms at the frequency observed in that user with EEG or another form of brain recording.

In some embodiments, one or more dominant tACS frequencies are chosen such that electrical coupling is more effective or optimal for one or more cell types (pyramidal neurons, interneurons, glial cells, or other cell types) based on their membrane time constants, ion channel kinetics, or other biophysical property. In other embodiments, one or more dominant tACS frequencies are chosen such that coupling is optimal for a subcellular compartment such as the dendrite, axon hillock, cell body, or synapse.

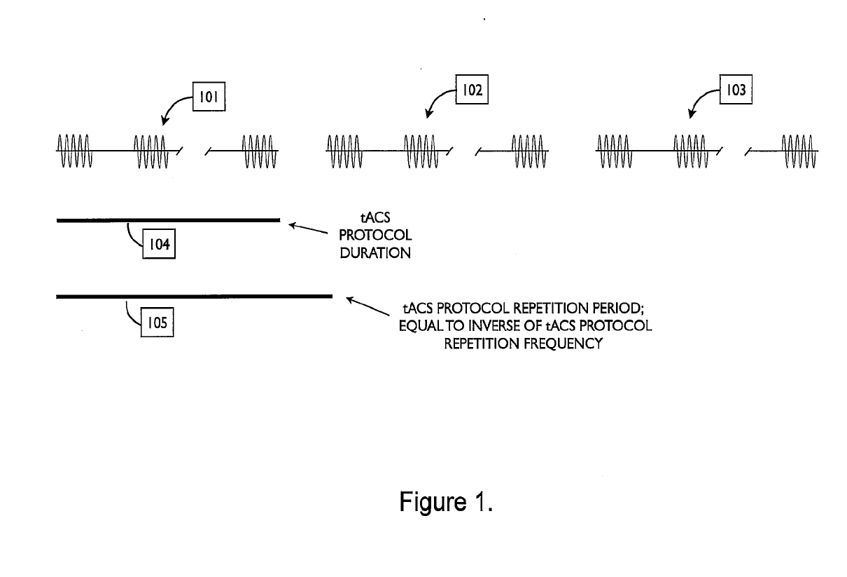

In many embodiments, electrical stimulation is pulsed as shown in FIG. 1. Pulsing TES is an effective strategy for inducing neuromodulation. Pulsed TES can use tDCS and/or tACS. Particularly advantageous pulsing strategies use pulsed tACS and deliver a TES protocol of two or more pulses 101 chosen from the group including but not limited to: about more than 2 pulses, about more than 3 pulses, about more than 4 pulses, about more than 5 pulses, about more than 10 pulses, about more than 20 pulses, about more than 50 pulses, about more than 100 pulses, about more than 500 pulses, about more than 1000 pulses, about more than 10000 pulses, or more pulses. The inter-pulse time and the number of pulses determine the TES protocol duration 104. In some embodiments, a pulsed TES protocol is repeated 102, 103 at a TES protocol repetition frequency 105 chosen from the group including but not limited to: about more than 0.001 Hz, about more than 0.01 Hz, about more than 0.1 Hz, about more than 1 Hz, about more than 5 Hz, about more than 10 Hz, about more than 20 Hz, about more than 50 Hz, about more than 100 Hz, about more than 250 Hz, about more than 500 Hz, about more than 1000 Hz, or faster. In some embodiments the pulse repetition rate is modulated during a TES session. In some embodiments, the pulse repetition rate is specific to a subset of one or more electrodes. Different electrodes or subsets of electrodes are pulsed with different repetition rates. Similarly, in some embodiments different electrodes or subsets of electrodes are driven at different frequencies and/or with different amplitudes.

Computational models are advantageous for modeling the transmission of electric fields in the brain. Effective computational models account for differential field shaping effects of different tissue types (e.g. skin, skull, white matter, grey matter, etc.) to derive an accurate estimate of induced electric fields.

A finite element model (FEM) is an advantageous embodiment of a computational model for estimating electric fields in the brain and can be used to determine the number, location, size, and shape of stimulating electrodes to use. Recent research and disclosures have described workflows and related methods for FEM of electric fields in the brain (e.g. U.S. patent application Ser. No. 13/294,994 in the name of Bikson et al., the entire disclosure of which is incorporated herein by reference). The FEM also determines stimulation parameters for each electrode (if there is a single reference electrode) or pair of electrodes (if multiple reference electrodes are used) in order to create a focused electric field in a brain region of interest. FEM models can be configured to optimize for both intensity and direction of current with a particular spatial and temporal profile. Both the strength and direction of an induced electric field determine the neuromodulation that occurs. The direction of an electrical field most significantly affects neuromodulation of white matter.

A workflow for FEM simulations is based on individualized head models created from magnetic resonance images. The pipeline starts by extracting the borders between skin, skull, cerebrospinal fluid, gray and white matter. The quality of the resulting surfaces is subsequently improved, allowing for the creation of tetrahedral volume head meshes that can finally be used in the numerical calculations. The pipeline integrates and extends established (and mainly free) software for neuroimaging, computer graphics, and FEM calculations into one easy-to-use solution.

In many advantageous embodiments, FEM electric field calculations are pre-computed for different electrode montages for the Standard Model or for a personalized anatomical model. By calculating the electric field computations for a given anatomy and electrode positioning before a targeted TES session, the necessary stimulation parameters can be optimized in significantly less computational time for different targeted brain regions. In this way, a preselected set of electrode montages and the associated electric field calculation is stored in a database either remotely or locally as a component of the device, thus permitting relatively rapid changes to the brain region targeted by changing the stimulation parameters delivered through the electrodes at known locations on the user's head. This feature of the device is advantageous for shifting targeting in the brain to achieve a desired form of neuromodulation.

In some embodiments, an optimization algorithm optimizes electrode positions and currents for a search space that includes one or more of: electrode positions and maximum and/or minimum currents at the electrodes, electrode size, and electrode shape. The optimization maximizes the electric field in a certain brain area and minimizes field strength at surrounding regions to achieve desired focality. While the number of simulated electrodes could comprise up to about 40 electrodes or more, a technical feasible reduction to about 8 electrodes could be made by selecting those electrodes which account for most of the variance in the electric field distribution over the electrodes using principal component analysis or a similar computational technique for dimensional reduction. In various embodiments, the number of electrodes used is chosen from the group including but not limited to: more than 2 electrodes, more than 3 electrodes, more than 4 electrodes, more than 5 electrodes, more than 7 electrodes, more than 10 electrodes, more than 15 electrodes, more than 25 electrodes, more than 50 electrodes, more than 100 electrodes, more than 500 electrodes, more than 1000 electrodes, more than 5000 electrodes, or more than 10000 electrodes. In some embodiments, a system comprises a processor configured to compute an optimization algorithm to optimize electrode positions and currents for a search space that includes one or more of: electrode positions and maximum and/or minimum currents at the electrodes, electrode size, and electrode shape.

In some embodiments, the spatial focus of electrical stimulation is selected, then electrode positions, shapes, or stimulation parameters are optimized to achieve this focus. In alternative embodiments, the direction or orientation of the electric field in the brain is selected, then electrode positions, shapes, and/or stimulation parameters are optimized to achieve this directionality. Some embodiments are optimized for both spatial location and the orientation of the electric field. In some embodiments, the electric field is optimized such that current flow is optimal with respect to the orientation of neuronal elements such as axon tracts which are measured by DTI tractography or estimated based on published anatomy.

In some embodiments, an FEM model uses an idealized spherical model of the head. In other embodiments, FEM based on anatomy of an individual is used to make a personalized and more accurate estimate of electric fields induced by TES. In these embodiments, the accuracy, localization, and/or effectiveness of targeted TES is improved due to the FEM being based on actual anatomy of the subject.

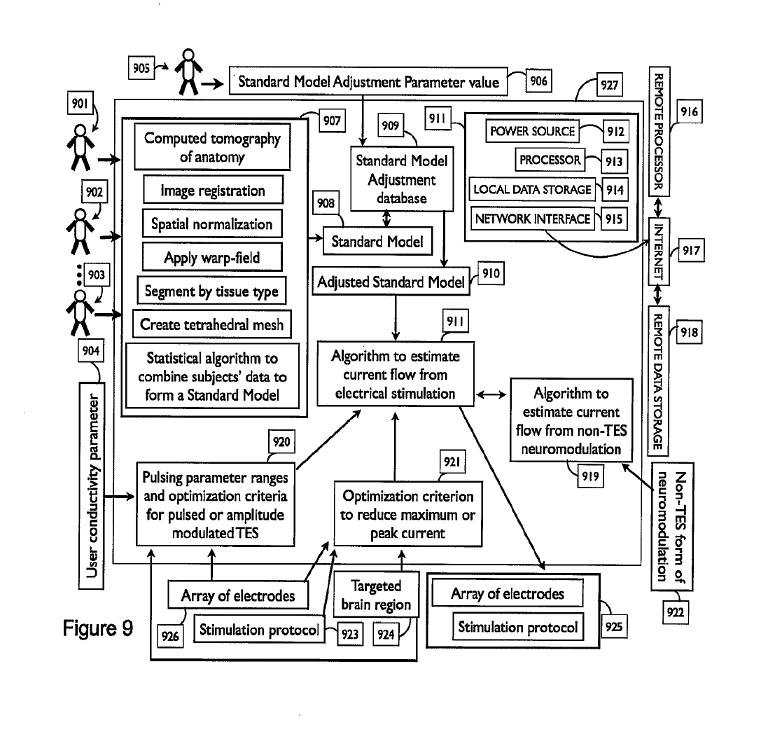

FIG. 9 shows method for the generation and usage of accurate individual head models in FEM based on MRI images suitable for incorporation in accordance with embodiments as disclosed herein. The method can comprise three main steps: a workflow for the generation and usage of accurate individual head models in FEM based on MRI involves three main steps (FIG. 9): (1) mesh generation; (2) field calculation; and (3) post-processing. Mesh generation comprises one or more of several steps, including: segmentation of different tissue types 1202, 1203 on the MR images 1201; generation of surfaces at the boundaries of each tissue type; rejoining of the hemispheres 1204; resolution of overlaps and artifacts; generation of a tetrahedral volume mesh; and, finally, decoupling 1205 and optimization of the mesh 1206. An optional step for mesh generation is inclusion of anisotropic conductivity values for white matter and grey matter based on diffusion tensor imaging (DTI). Field calculation uses the anatomical mesh and the location and number of TES inputs to determine the fields generated. Optional post-processing scripts can be used to conveniently convert file formats, extract regions or elements, and compare models across conditions or users.

Although the MR images may comprise an image of one individual, in many embodiments a plurality of images are input in order to provide data of a normal subject. This data of the normal subject is used to generate a standard mesh that can be adjusted based on user data as described herein.

At a step 1210, reference data of patient parameters as described herein are output. The patient parameters that are output may correspond to dimensions of a patient or the patient population used to derive the volumetric meshing and optimization that is adjusted to the user, for example with one or more of scaling, rotating, shifting or warping of the mesh to fit the subject.

At a step 1207, a user inputs data. The data may comprise data about the subject, and the subject may comprise a user, for example when the user treats himself (or herself).

At a step 1208, subject data is compared with reference data. The reference data may comprise data of a patient population.

A volume mesh meshing and optimization step can be performed, and the mesh passed to a step 1209.

At a step 1209 the volume mesh is adjusted in response to the user data. The shape of the mesh can be mapped or scaled, for example, such that the mesh fits the subject input data, for example fits external dimensional data of the subject. For example, several externally measurable patient metrics as described herein can be used to adjust the mesh. Adjustment of the mesh may comprise adjustment of the elements of the finite element array so as to fit the patient data. In many embodiments, each of the plurality of finite elements comprises one or more nodes that define said each element.

The adjusted model may comprise an adjusted finite difference model, for example, with nodes adjusted similarly to the FEM as described herein.

In many embodiments, a system comprises a processor configured to compute a FEM model to estimate current densities in the brain due to stimulation from two or more TES electrodes.

In many embodiments, a system comprises a processor configured to compute a FEM model that selects electrode locations to be on regions of the head, face, neck, or other body area that do not have hair.

The method of FIG. 9 is shown in accordance with many embodiments. Those of ordinary skill in the art will recognize many alternative, complementary, and supplementary embodiments. The steps can be removed, replaced or repeated. The steps may comprise sub-steps. The steps can be performed in any order. The processor of the system disclosed herein can be configured to perform one or more of the steps of the method. The processor may comprise instructions of a computer readable that embody instructions of a computer program to implement an algorithm, for example. For example, computational models and workflow components as described herein can be advantageously employed for mapping electric fields in the brain in accordance with the present disclosure.

In this disclosure, the term Standard Model refers to an anatomical model of the head that captures common anatomical features and is reasonably or acceptably accurate for use across individuals. In some embodiments, the Standard Model is generated by normalizing and/or averaging anatomical maps of the head and brain.

In some embodiments, a processor is configured to run an FEM based on a Standard Model of a human head and brain and the results are used to estimate electric fields generated by a pattern of electrical stimulation—and, conversely, the pattern and location of electrical stimulation required to target a particular location in the brain with a particular array or set of stimulating electrodes.

In various embodiments, a Standard Model is adjusted for a user based on one or more values of parameters from the group including but not limited to: white matter tracts as measured by diffusion tensor imaging (DTI), grey matter regions, age, gender, height, weight, or other demographic, health, or behavioral assessment. In some embodiments, the computational model includes parameters that account for the biophysical properties of hair, skin, skull, dura, brain, and other tissues that are affect or otherwise modify transmitted electrical fields. In some embodiments, the computational model is automated or semi-automated and can be controlled by the user for targeting one or more of their brain regions.

Embodiments that use an adjusted Standard Model do not require that a user undergo an MRI session. An FEM or other suitable computational model can be computed more efficiently if the mesh generation and/or field calculation steps can be pre-computed. In many embodiments, an apparatus for estimating a current induced in the brain by transcranial electrical stimulation treatment of a subject comprises: a standard model of brain and head anatomy based on a structural scan of an individual and stored in a computer readable memory; a database or lookup table indicating adjustments to make to a standard model of brain and head anatomy based on at least one Standard Model Adjustment Parameter; and a processor configured to load a standard model of brain and head anatomy from a computer readable memory, determine one or more standard model adjustments to make by querying a database or lookup table, and compute an adjusted standard model of brain and head anatomy.

In embodiments, Standard Model Adjustment Parameters are selected from the group including but not limited to: an anatomical measurement of a user's skull, scalp, hair, face, head, dura, brain, neck, or other part of the body; a cognitive assessment that takes the form of one or more of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task; a physiological measurement of the body that takes the form of one or more measurements chosen from the group including but not limited to: electromyogram (EMG), galvanic skin response (GSR), heart rate, blood pressure, respiration rate, electrocardiogram (EKG), pulse oximetry (e.g. photoplethysmography), heart rate, pupil dilation, eye movement, gaze direction, and other physiological measurement known to one skilled in the art; gender, height, weight, age, diet, pharmaceutical drugs used, cognitive abilities, cognitive disabilities, or other metadata; a genetic aspect of a user including but not limited to: microduplication, microdeletion, single nucleotide polymorphism (SNP), aneuploidy, allele, or other genetic data.

In an advantageous embodiment of the disclosure, adjustments to the Standard Model are computed by a processor component on a remote server using data about the Standard Model and adjustment parameters stored in a computerized memory on a remote server. In some embodiments, the processor is further configured to write the adjusted standard model of brain and head anatomy to a computer readable memory. In some embodiments, the apparatus further comprises a communication system for transmitting information between a remote processor and a TES system controller. In some embodiments, a TES system controller may be a microcontroller or microprocessor component of a wearably attached and/or portable TES system. In alternative embodiments, the microcontroller processor is a smartphone, tablet computer, or other computerized system that can communicate using a wired or wireless communication protocol. In many embodiments, a user, automated system, or third party individual transmits one or more Standard Model Adjustment Parameters values via the Internet or another standardized computerized communication framework to a remote processor where adjustments to the Standard Model are computed. In some embodiments, the user, automated system, or third party individual also transmits one or more TES System Parameters selected from the group including but not limited to: firmware version, number of electrodes, location of electrodes, size and shape of electrodes, stimulation protocol history, capacity of the system to deliver direct current stimulation and/or alternating current stimulation, battery charge remaining, maximum current deliverable, constraints on anode-cathode pairs that can be created from available electrodes, and other information about a TES system. In some embodiments, the user, automated system, or third party individual also transmits one or more brain region targets. In some embodiments, the user, automated system, or third party individual also transmits data concerning the outcome of one or more previous TES sessions, where the transmitted data is selected from the group including but not limited to: a subjective assessment by the user or another individual, a cognitive assessment, a brain recording or other physiological measurement, or other outcome assessment.

Many embodiments comprises a processor configured to adjust a Standard Model based on one or more Standard Model Adjustment Parameters and/or one or more TES System Parameters.

In many embodiments, the processor is further configured to estimate induced current density and direction in one or more brain regions based on a TES stimulation protocol, then transmit a result of the adjusted Standard Model to the user or another system or individual. This embodiment of the disclosure provides feedback about which brain region was targeted by TES.

In an alternate embodiment of the disclosure, the processor is further configured to optimize one or more TES stimulation parameters to target one or more brain regions, then transmit instructions to a TES system controller to adjust one or more parameters chosen from the group including but not limited to: an electrode position; anode-cathode pairing of two or more electrodes; current delivered from an anode-cathode pair of electrodes; timing of stimulation from electrodes; frequency of alternating current stimulation; or other TES parameter. This embodiment of the disclosure enables improved targeting of TES.

Some embodiments comprise one or more Standard Models stored in a computer-readable memory. In embodiments the one or more Standard Models are stored locally as part of a TES system or stored remotely and accessible via the Internet. An embodiment comprises a processor configured to select an appropriate Standard Model for an individual based on the values of one or more Standard Model Adjustment Parameters. This feature of the disclosure provides improved fit for an individual's anatomy with little additional memory storage or computational work required relative to having a single Standard Model. This feature of the disclosure is similar to the way that different sizes of clothing permit individuals with a range of body types to find clothes that fit without the cost of custom tailoring.

The mesh model of the head and brain used for the FEM process is an important aspect of the field mapping process. In some embodiments, a personalized anatomical map is generated for each user after an MRI session.

In many embodiments, targeting is personalized based on structural imaging of a user's head and brain. For instance, a user's skull thickness affects the transmission of an electric field. Magnetic resonance imaging (MRI) is effective for mapping anatomy with sufficient accuracy and sensitivity. MRI protocols for diffusion tensor imaging (DTI) are particularly advantageous. DTI provides anatomical data about white matter tracts between brain regions. In some embodiments, conductivity anisotropy from diffusion weighted MRI images is used to improve the realism of the computational model of electric fields.

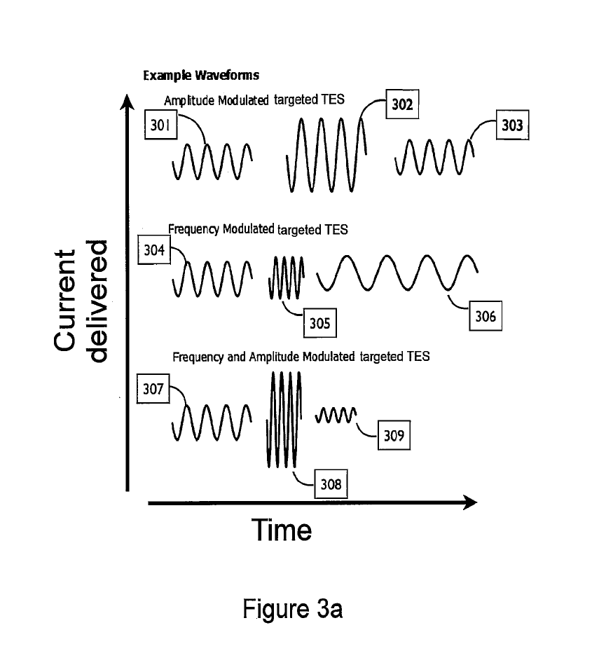

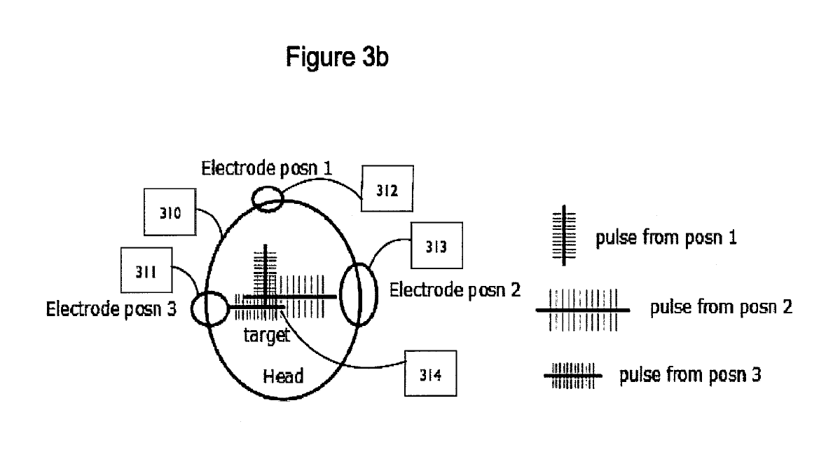

In some embodiments wherein the device is configured for pulsed tACS, the amplitude of tACS pulses comprise one or more amplitude modulated pulses 301, 302, 303, as shown in FIG. 3A. In alternative or combinational embodiments wherein the device is configured for pulsed tACS, the frequency of tACS pulses is modulated and the pulses comprise one or more frequency modulated pulses 304, 305, 306. In many embodiments wherein the device is configured for pulsed tACS, the amplitude and frequency of tACS pulses comprise frequency and amplitude modulated pulses 307, 308, 309, for example.

In human subjects, transcranial TES via a plurality of electrodes on a user's head 310 are targeted to one or more discrete brain regions, including brain regions deep below the surface of the brain and skull 314. Targeted TES can be achieved by using an array of multiple electrodes 311, 312, 313 and passing current through electrodes at each position (‘posn’ in FIG. 3b) with appropriate parameters selected from the group including but not limited to: timing, duration, frequency (in some embodiments including direct current stimulation), pulse repetition, intensity, and phase. Those of ordinary skill in the art will recognize that FIG. 3 shows an embodiment of how three or more electrodes can achieve spatially restricted electric fields deep in the brain through constructive and destructive interference and is not meant to restrict the number, positioning, size, or other feature of TES electrodes in this disclosure.

Constructive and destructive interference between currents delivered from three or more sets of electrodes can affect the density and direction of currents induced in the brain. This feature can be leveraged to generate spatially restricted regions of electrical stimulation in the brain by delivering phase-shifted electrical stimulation from three or more sets of electrodes. Due to slight propagation delays of electrical fields in biological tissue, the spatial location of current density and direction can be shaped by changing delays between phase-shifted electrical pulses delivered from electrodes at different locations on the head, face, neck, or elsewhere on the body. Systems configured to have multiple electrode pairs pulsed at defined latencies relative to each other can be used to target electrical stimulation to one or more brain regions. Systems that target deep brain regions based on interference patterns can be constructed with TES systems configured for pulses of direct current stimulation, alternating current stimulation, or both direct current stimulation and alternating current stimulation. Advantageous pulsing regimes incorporate phase shifts between pulses from different sets of electrodes of generally less than 10 ms, often less than 1 ms, optionally less than 100 microseconds, and optionally less than 1 microsecond.

Many embodiments comprises a processor configured to compute an FEM or other suitable computational model to determine TES stimulation parameters for three or more electrodes configured to deliver phase-shifted pulses of direct current stimulation to target deep brain regions through interference of the multiple transmitted electric fields. An alternative embodiment of the disclosure comprises a processor configured to compute an FEM or other suitable computational model that determines the TES stimulation parameters for three or more electrodes configured to deliver phase-shifted alternating current stimulation to target deep brain regions through interference of the multiple transmitted electric fields.

In further embodiments, the computational model configured to be user actuated user requires that the user select one or more from the group including but not limited to: the number of electrodes through which to deliver TES; the one or more brain regions to target; the duration of the TES session; the range of intensity of TES employed; the range of pulse repetition frequencies to use; the range of alternating current frequencies to use; the modulation of TES parameters; the number of repetitions of a TES Protocol; and the frequency of repetition of a TES protocol.

In some embodiments, the TES protocol is adjusted based on demographic or other metadata of the user chosen from the group including but not limited to: gender, height, weight, age, diet, pharmaceutical drugs used, cognitive abilities, cognitive disabilities, or other metadata. The TES protocol adjustment based on metadata includes parameters from the list including but not limited to: targeted brain regions; the placement of electrodes; the number of electrodes; shape of electrodes; other property of electrodes that relates to stimulation; use of DCS for one or more pairs of electrodes; use of ACS for one or more pairs of electrodes; frequency of ACS stimulation; intensity of stimulation; timing of stimulation; and modulation of any stimulation parameter during the TES protocol.

In some embodiments, the TES protocol is adjusted based on anatomical measurements of a user's skull, scalp, hair, face, head, dura, brain, neck, or other part of the body. In other embodiments, the TES protocol is adjusted based on a cognitive assessment that takes the form of one or more of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task.

In yet other embodiments, the TES protocol is adjusted based on a physiological measurement of the body that takes the form of one or more measurements chosen from the group including but not limited to: electromyogram (EMG), galvanic skin response (GSR), heart rate, blood pressure, respiration rate, electrocardiogram (EKG), pulse oximetry (e.g. photoplethysmography), heart rate, pupil dilation, eye movement, gaze direction, and other physiological measurement known to one skilled in the art.

In alternative embodiments, the computational model is configured by a skilled practitioner. In these embodiments, the skilled practitioner select one or more from the group including but not limited to: the number of electrodes through which to deliver TES; the one or more brain regions to target; the duration of the TES session; the range of intensity of TES employed; the range of pulse repetition frequencies to use; the range of alternating current frequencies to use; the modulation of TES parameters; the number of repetitions of a TES Protocol; and the frequency of repetition of a TES protocol.

In some embodiments, the placement of electrodes is adjusted based on a procedure that delivers a test pulse of known electrical current through one or more electrodes and measures the induced electric field.

In various embodiments, the device or system is configured for a user to deliver targeted TES to themselves; for someone to deliver the TES protocol to a subject; or for a skilled practitioner such as nurse, doctor, therapist, or other trained expert to deliver targeted TES to a subject.

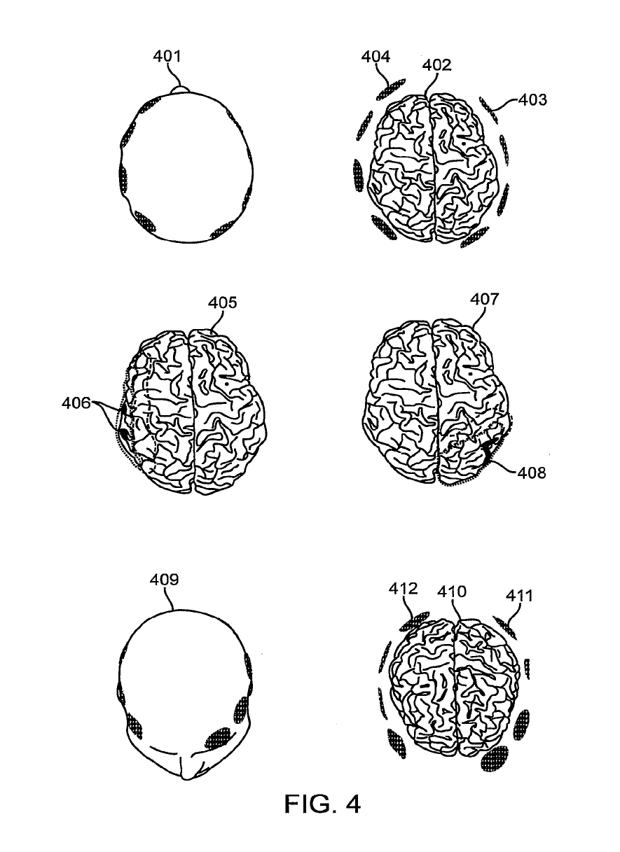

In various embodiments, the device or system is configured so that the induced neuromodulation is perceived subjectively by the recipient as a sensory perception, movement, concept, instruction, other symbolic communication, or modifies the recipient's cognitive, emotional, physiological, attentional, or other cognitive state. An example of using FEM to estimate electric fields caused by specific patterns of current delivered through an array of electrodes is illustrated in FIGS. 4, 5, and 6. In this embodiment, the head model was created from T1 and T2 weighted MR images to generate a model of the skull and skin 401, 409, as well as the brain 402. This was done with a commercially available software package. In some embodiments, the head model is improved by incorporating other anatomical features that have distinct conductivity properties such as the eyeballs, tongue, and other structures in the head, neck, and elsewhere in the body.

A set of eight electrodes was selected and these were modeled to be placed around the circumference of the model head and brain 402, 410. The eight electrodes were numbered (1-8) radially around the head with 403, 411 as electrode 1 and 404, 412 as electrode 8 on the two views of the model head shown at the top and bottom rows of FIG. 4.

In this example, the goal was to target the left temporal lobe 406 and the right occipital cortex 408 as indicated by a map of electric field intensity in the modeled brain 405, 407. Effective focal targeting was achieved by preselecting a desired spatial target, then optimizing the currents transmitted through each of the eight electrodes to create a spatially constrained area of electrical stimulation.

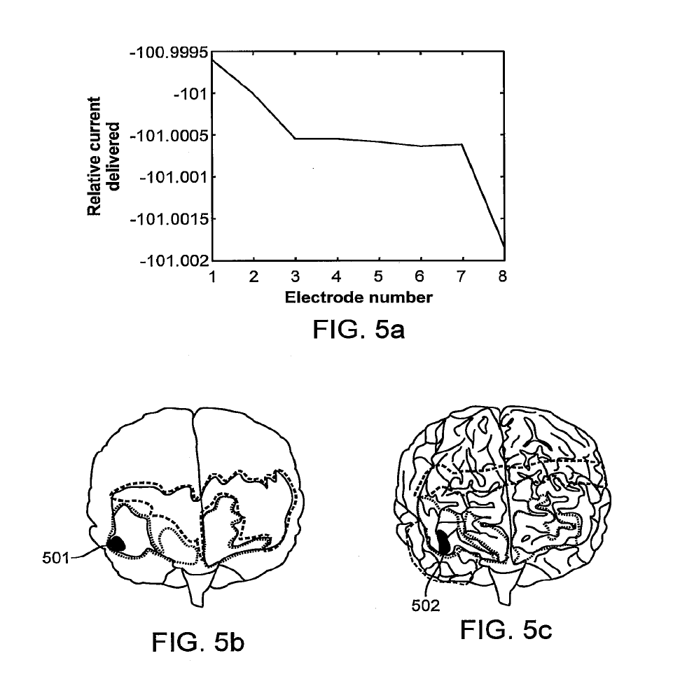

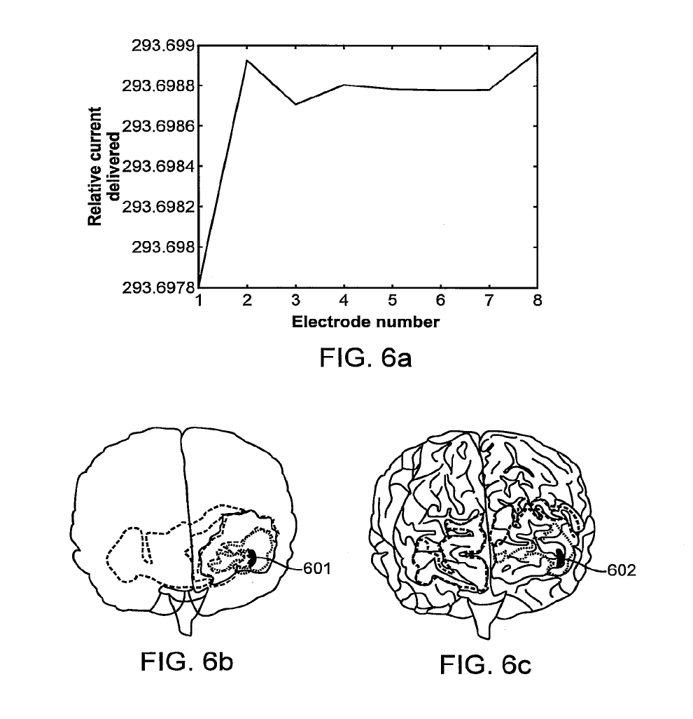

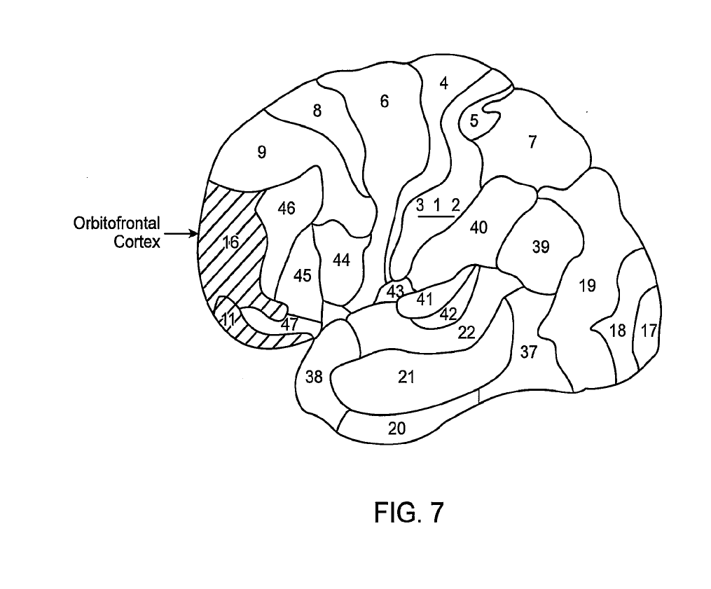

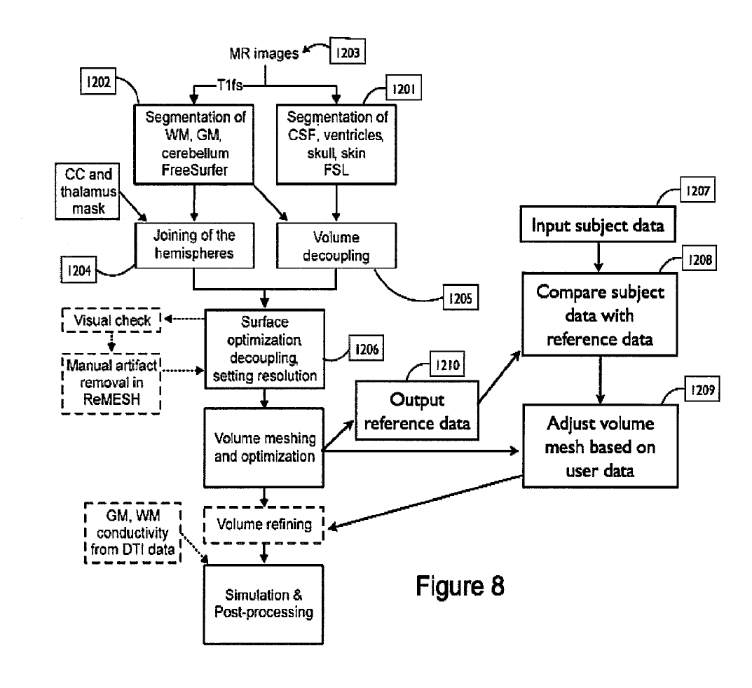

Using the same anatomical model as in FIG. 4, new targets were selected: left prefrontal cortex (PFC) and right PFC. The same eight-electrode arrangement was used, so the optimization process was computationally efficient. Similar to the results for the temporal and occipital cortices, spatially restricted foci were achieved in either right PFC (FIG. 5) or left PFC (FIG. 6) as indicated by a map of electric field direction 501, 601 and focal intensity to identify focal spots 502, 602 in the target location of the modeled brain. The electrode nearest the focal spot is expected to deliver the highest relative current, and this is corroborated by plots showing relative current delivered vs. electrode number at the top of FIGS. 5 and 6.

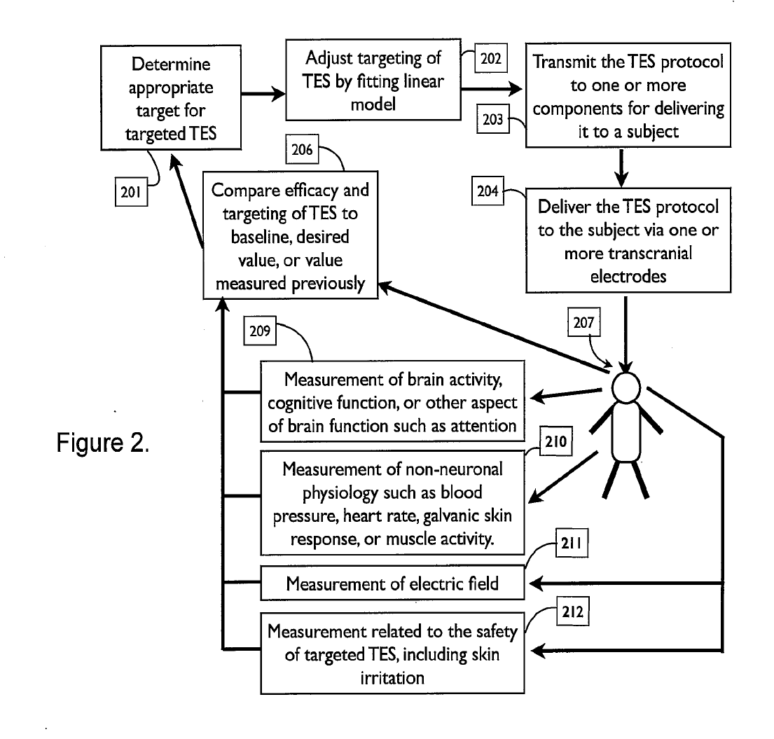

A schematic description of embodiments of the disclosure is shown in FIG. 2. In these embodiments of the disclosure, the device is configured in accordance with a method so as to operate in a closed loop manner to improve the specificity of the desired neuromodulation. Targeting of the TES is adjusted based on one or more measurements that assess the effect of stimulation.

An estimate of targeting for TES is determined 201, and the pattern of electrical stimulation for a given electrode montage is optimized 202 based on a pre-computed FEM for a given anatomical model and electrode montage. The electrode positioning is communicated to the user, another individual, or automatically to a component of the system so that electrodes can be properly placed, and the stimulation protocol is transmitted 203 to device components for delivering TES 204 and the TES protocol is delivered to the subject 207. Efficacy of TES can be assessed by one or more of: measurement of brain activity, cognitive function, or other aspect of brain function such as attention 209; measurement of non-neuronal physiology such as blood pressure, heart rate, galvanic skin response, or muscle activity 210; measurement of the induced electric field 211; and measurements related to the safety of TES 212. The efficacy and actual targeting of TES are compared to baseline, a desired value, and/or a value previously measured 206 to determine whether targeting of TES should be adjusted 201.

In embodiments of the disclosure, the system is configured to induce neuromodulation in a user that is perceived subjectively by the user as a sensory perception, movement, concept, instruction, other symbolic communication, or modifies the user's cognitive, emotional, physiological, attentional, or other cognitive state. In these various embodiments, one or more appropriate brain regions are selected to achieve a specified neuromodulatory effect, then TES is targeted to these brain regions based on a FEM or other computational model that estimates electric fields in the brain. In some embodiments, the system is configured for use in non-clinical settings and may also be configured to be user-actuated or automated.

In many embodiments, targeted TES is combined with other neuromodulatory stimulation techniques to achieve effects in the brain. These embodiments are advantageous for neuromodulation that may not be possible with either effect by itself. Other brain stimulation modalities include transcranial ultrasound neuromodulation, transcranial magnetic stimulation (TMS), deep brain stimulation (DBS), optogenetic stimulation, one electrode or an array of electrodes implanted on the surface of the brain or dura (electrocorticography (ECoG) arrays), radio-frequency stimulation, and other modalities of brain stimulation known to one skilled in the art.

In an embodiment for estimating a current induced in the brain by transcranial electrical stimulation treatment of a subject comprises a standard model of brain and head anatomy based on a structural scan of an individual and stored in a computer readable memory; a processor configured to compute a computational model for estimating current density and direction in the brain; and components to deliver brain stimulation by one or more techniques other than TES chosen from the group consisting of: transcranial ultrasound neuromodulation, transcranial magnetic stimulation (TMS), deep brain stimulation (DBS), optogenetic stimulation, one electrode or an array of electrodes implanted on the surface of the brain or dura (electrocorticography (ECoG) arrays), radio-frequency stimulation, and other modalities of brain stimulation known to one skilled in the art.

In some embodiments, the components for brain stimulation by one or more techniques other than TES is triggered with a pre-defined temporal relationship relative to a TES protocol. In other embodiments, the brain stimulation by one or more techniques other than TES is delivered concurrently with a TES protocol.

In many embodiments, neuromodulation is targeted to more than one brain region. In some embodiments, targeted TES or another technique for neuromodulation targets a first brain region to induce a set of behavioral, cognitive, or other effects, while concurrently (or in close temporal relation) targeting a second brain regions to counteract a subset of the effects of stimulation targeting the first brain region. In this manner, the functional effect of neuromodulation can be shaped to reduce unwanted side effects. In some embodiments that target multiple brain regions, the brain regions are anatomically nearby brain regions. In other embodiments that target multiple brain regions, the brain regions are anatomically distant brain regions.

In some embodiments, multiple brain regions are targeted that relate to language processing in order to facilitate or enhance language learning. In some embodiments, multiple brain regions are targeted that relate to decision making in order to modulate decision making. In advantageous embodiments that affect decision making, brain recording is used to detect incipient mistakes in real-time by neuromodulation induced by targeted TES or another form of brain stimulation. In some embodiments, TES is targeted to reduce activity in the left parietal cortex and increase activity in the right parietal cortex in order to enhance creativity. In some embodiments, TES is targeted to multiple sites that play a role in visual processing to affect how visual inputs are perceived.

In some embodiments, the device is configured to target TES to multiple brain regions with a pre-defined temporal relationship. In some embodiments in which multiple brain regions are targeted with a pre-defined temporal relationship, the device is configured to target multiple brain regions concurrently. In some embodiments in which multiple brain regions are targeted with a pre-defined temporal relationship, the device is configured to target multiple brain regions with a specific latency between stimulation targeting each of the brain regions. In some embodiments in which multiple brain regions are targeted with a pre-defined temporal relationship, the device is configured so that the latency for stimulation between multiple brain regions is determined by the natural neuronal conduction velocity between the targeted brain regions.

In some embodiments in which multiple brain regions are targeted with a pre-defined temporal relationship, the device is configured to target a first brain region and a second brain region to counteract an unwanted effect occurring in or mediated by the second brain region caused by stimulation of the first region. In some embodiments in which multiple brain regions are targeted with a pre-defined temporal relationship, the device is configured to target additional brain regions to counteract the effects of stimulating a first and/or second brain region. In some embodiments in which multiple brain regions are targeted with a pre-defined temporal relationship, the device is configured for concurrent stimulation of the first and second brain regions. In some embodiments in which multiple brain regions are targeted with a pre-defined temporal relationship, the device is configured such that stimulation of the first and second brain regions occurs with a specified latency, where the latency is chosen from the group including but not limited to: less than about 30 seconds; less than about 10 seconds; less than about 5 seconds; less than about 1 second; less than about 500 milliseconds; less than about 250 milliseconds; less than about 100 milliseconds; less than about 50 milliseconds; less than about 40 milliseconds; less than about 30 milliseconds; less than about 20 milliseconds; less than about 10 milliseconds; less than about 5 milliseconds; less than about 2 milliseconds; or less than about 1 millisecond.

In some embodiments in which multiple brain regions are targeted with a pre-defined temporal relationship, parameters of stimulation of multiple brain regions and relative timing of stimulation are determined based on feedback from a measurement of brain activity, behavior, cognition, sensory perception, motor performance, emotion, or state of arousal.

In some embodiments, the device is configured to induce spike-timing dependent plasticity in one or more targeted brain regions. In some embodiments for inducing spike-timing dependent plasticity, the device is configured to re-create patterns of neural activity in and/or between distinct brain regions during which transduction delays of between about 1 ms and about 30 ms occur.

In some embodiments, random noise stimulation is delivered. Random noise stimulation has been shown to induce neuroplasticity. Advantageous embodiments that use random noise stimulation delivered by TES target specific brain regions for neuroplasticity or broader areas as large as a cortical hemisphere or the entire brain.

In many embodiments, the timing of targeted TES is designed to modulate brain activity that occurs in the temporal domain. Inembodiments of the disclosure, TES is used to activate, inhibit, or modulate brain rhythms in one or more brain regions. In another embodiment of the disclosure, TES is targeted to multiple connected regions in the brain that normally communicate with a known temporal latency. By stimulating multiple brain regions with TES and/or another technique for neuromodulation, communication or coupling between disparate brain regions can be enhanced, disrupted, phase-shifted or otherwise modulated.

In many embodiments, brain recordings are used to measure the effect of targeted TES. This technique is advantageous for providing feedback (in some embodiments, real-time feedback) concerning the targeting, timing, and stimulation parameters for targeted TES and/or other techniques for neuromodulation used. In this embodiment of the disclosure, the measurement of brain activity takes the form of one or a plurality of: electroencephalography (EEG), magnetoencephalography (MEG), functional magnetic resonance imaging (fMRI), functional near-infrared spectroscopy (fNIRS), positron emission tomography (PET), single-photon emission computed tomography (SPECT), computed tomography (CT), functional tissue pulsatility imaging (fTPI), xenon 133 imaging, or other techniques for measuring brain activity known to one skilled in the art.

In some embodiments, the effect on the brain is measured by a cognitive assessment that takes the form of one or more of: a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task.

In many embodiments, physiological monitoring is used to measure the effect of targeted TES. This technique is advantageous for providing feedback (in some embodiments, real-time feedback) concerning the targeting, timing, and stimulation parameters for targeted TES and/or other techniques for neuromodulation used. In this embodiment of the disclosure, the measurement of physiological signals takes the form of one or a plurality of: electromyogram (EMG), galvanic skin response (GSR), heart rate, blood pressure, respiration rate, electrocardiogram (EKG), pulse oximetry (e.g. photoplethysmography), pupil dilation, eye movement, gaze direction, or other physiological measurement known to one skilled in the art.

In many embodiments, a device assists a user or other individual in placing electrodes at appropriate locations to achieve a desired form of neuromodulation. Methods for guiding the user or other individual to place electrodes at the one or more desired locations includes one or more from the group including but not limited to: fiduciary markers on the head; ratiometric measurements relative to fiduciary markers on the head; alignment components that detect relative location of electrode components by proximity as measured by radiofrequency energy, ultrasound, or light; or a grid or other alignment system, such as the position of the electrodes themselves, projected onto the head of the user. In some embodiments, an indicator provides feedback when the electrode positioning is achieved through a light-, sound-, or tactile-based indicator.

In some embodiments, a user or other individual identifies fiduciary markers to assist in targeting. Fiduciary markers on the head include those used for placing EEG electrodes in the standard 10/20 arrangement. The nasion is the point between the forehead and the nose. The inion is the lowest point of the skull from the back of the head and is normally indicated by a prominent bump.

In many embodiments, neuromodulation is achieved exclusively via electrodes placed on portions of the head that do not have hair to reduce the need for additional material or system components for coupling the electrical current to the scalp. Targeted TES is achieved with a system that includes one or more electrodes placed on hairless portions of the head, face, and neck. In some embodiments, an electrode placed on the periphery (below the neck) is used to deliver a spatially broad electrical field to the brain.

In many embodiments, coupling between a stimulating electrode and the skin is achieved with a semi-permeable sack between the electrode and the skin that releases a small amount of water or other conductive liquid when squeezed. In some embodiments of this aspect of the disclosure, the water or other conductive liquid evaporates after the TES session and does not require cleanup.

In many embodiments, an array of TES electrodes is present on a single unit placed on the head and contains components for bussing between pairs of electrodes. These device components enable very high density electrodes to be used for improved targeting of TES while reducing the number of individual assemblies that a user must affix or stick to their head.

In many embodiments, the system is portable and battery powered. In some embodiments, the battery is charged by one or more of solar panels or by harvesting energy from the movements of a user for example by using piezopolymers or piezoelectric fiber composites.

In core embodiments, the disclosure comprises hardware and/or software components that generate appropriate control sequences for TES, transmit them to current or voltage stimulator hardware, and connect to electrodes placed on a user for generating electrical currents. In some embodiments, the system is configured for mobile use. In some embodiments, the system is configured for wireless communication to a base station or via cellular networks to the Internet. In some embodiments, the system further comprises one or more components for electrical isolation of the stimulating electrodes (and user).

In many embodiments, the placement of TES electrodes and spatiotemporal pattern of stimulation delivered through the TES electrodes is configured for targeting the ventromedial prefrontal cortex for neuromodulation (VmPFC; Brodmann area 10). Targeting to the VmPFC can be advantageous for modulating emotion, risk, decision-making, and fear.

In many embodiments, the placement of TES electrodes and spatiotemporal pattern of stimulation delivered through the TES electrodes is configured for targeting the orbitofrontal cortex for neuromodulation (OFC; Brodmann 10, 11, 14). Targeting to the OFC can be advantageous for modulating executive control and decision making.

In many embodiments, the placement of TES electrodes and spatiotemporal pattern of stimulation delivered through the TES electrodes is configured for targeting the ventral striatum for neuromodulation. Targeting to the ventral striatum can be advantageous for modulating emotional and motivational aspects of behavior.

In many embodiments, the placement of TES electrodes and spatiotemporal pattern of stimulation delivered through the TES electrodes is configured for targeting the locus coeruleus for neuromodulation (LC). Targeting to the LC can be advantageous for modulating norepinephrinergic tone, learning and memory, sleep, processing of stressful stimuli, and other effects.

In many embodiments, the placement of TES electrodes and spatiotemporal pattern of stimulation delivered through the TES electrodes is configured for targeting the ventral tegmental area for neuromodulation (VTA). Targeting to the VTA can be advantageous for modulating reward circuitry, motivation, drug addiction, intense emotions relating to love, and other effects mediated by this dopaminergic system.

In some embodiments, targeted TES is used to affect, bias, or modulate brain activity by time-lapsing the generation and transmission of weak electric fields from pre-defined (modeled) electrode locations on a user's head based on desired outcome. In some embodiments, computational simulation of time-staggered (phased) weak electric fields is used to modulate environmental awareness.

In alternative embodiments, a subject does not undergo an MRI scan, and a Standard Model of head and brain anatomy is used for modeling electric fields in a user. In yet another embodiment, the Standard Model is adjusted based on head measurements such as circumference, eye distance, interaural distance, nasion, inion, or other head measurements known to one skilled in the art.

In some embodiments, one or more brain regions is targeted based on a database component of the system that determines appropriate targeting for a desired outcome. For example a user desires modulation of decision making by targeted TES. The database component of the system determines the appropriate one or more brain regions to target and calculates the appropriate electrode placement and stimulation parameters. In some embodiments, the appropriate stimulation parameters are transmitted automatically to the device components that control the timing, amplitude, and frequency of electrical stimulation. In other embodiments, the stimulation parameters are communicated to the user or other individual who enters them into a user interface component of the system or selects them from a pre-populated list in a user interface component of the system. In some embodiments, the database component of the system also includes a physiological event intended to occur in response to targeted TES. The intended response may be neurochemical, such as an increase in neurotransmitter levels; neurophysiological, such as by a change in activity in one or more brain regions; physiological, as measured by biological sensors that detect changes outside the central nervous system; or behavioral, as assessed with a cognitive test or evaluation.

In some embodiments, the one or more effects of using multiple forms of neuromodulation are chosen from the list including but not limited to: increasing the spatial extent of stimulation; decreasing the spatial extent of stimulation; reshaping the spatial extent of stimulation; modifying the nature of the induced neuromodulation; increasing the intensity of neuromodulation; decreasing the intensity of neuromodulation; mitigating a cognitive or behavioral affect; enhancing a cognitive or behavioral affect; modifying the cells affected by neuromodulation; modifying the cellular compartments affected by neuromodulation; or another modification of the neuromodulating energy transmitted into the brain and/or nervous system.

In some embodiments, computational modeling to determine electrode placement and electrode stimulation parameters occur remotely form the one or more device components wearably attached to the user. When the computational model for determining targeted TES electrode placement and stimulation parameters occurs remotely, it may occur at a centralized facility for instance a remote server and be transmitted to the device wearably attached to the user via the Internet. In alternative embodiments, device components wearably attached or near the user achieve the necessary computational modeling. In alternative embodiments, the user's head model may be stored locally for generation of multiple TES protocols in a user-specific manner.

In some embodiments, electrodes include a reflective surface in order to provide positioning relative to a person's head by way of feedback from an infrared (IR) or other optical sensor. In some embodiments, a small IR light emitting diode (LED) is used to provide a reflected light source to achieve electrode placement with respect to the user's head. Eye tracking may also be useful for providing feedback to the user of electrode placement relative to their gaze.

In various embodiments, electrode pad shape and size varies in order to achieve prescribed targeting.

In various embodiments, device components transmit pulsed or amplitude modulated or frequency modulated weak-electrical fields from different locations on the head such that constructive interference patterns provide an effective targeting of the electrical field.

In some embodiments, the device incorporates a built-in impedance meter. Advantageous embodiments provide the user with feedback about the impedance of each electrode (or electrode pair) to guide the user or other individual as to the effectiveness with which an electrode has been electrically coupled to their head. In various embodiments, feedback about electrode impedance is provided through one or more of: a graphical user interface, one or more indicator lights, or other user interface or control unit.

Combining targeted TES with transcranial ultrasound neuromodulation is advantageous for more effectively targeting the temporal and/or spatial extent of neuromodulation. Combining targeted TES with transcranial ultrasound neuromodulation is also beneficial for shaping the induced cognitive, behavioral, perceptual, motor, or other change in brain function. For instance, TES could be used to “clamp” shallow areas near the brain surface so that no change in brain function occurs during the transmission of ultrasound to a deeper brain region desired to be affected by transcranial ultrasound neuromodulation. In another embodiment of the disclosure that combines TES and transcranial ultrasound neuromodulation, supralinear enhancement of neuromodulation is achieved so that low energy levels to improve the safe operation of the system.

In some embodiments, a temporal sequence of input/output currents at the selected electrodes is computed that stimulates different brain regions in a defined temporal order to achieve a desired change in brain function.

In some embodiments, the system or device is configured to target one or more regions of cerebral cortex, where the region of cerebral cortex chosen from the group including but not limited to: striate visual cortex, visual association cortex, primary and secondary auditory cortex, somatosensory cortex, primary motor cortex, supplementary motor cortex, premotor cortex, the frontal eye fields, prefrontal cortex, orbitofrontal cortex, dorsolateral prefrontal cortex, ventrolateral prefrontal cortex, anterior cingulate cortex, and other area of cerebral cortex.

In other embodiments, the system or device is configured to target one or more deep brain regions chosen from the group including but not limited to: the limbic system (including the amygdala), hippocampus, parahippocampal formation, entorhinal cortex, subiculum, thalamus, hypothalamus, white matter tracts, brainstem nuclei, cerebellum, neuromodulatory nucleus, or other deep brain region.

In some embodiments, the system or device is configured to target one or more brain regions that mediate sensory experience, motor performance, and the formation of ideas and thoughts, as well as states of emotion, physiological arousal, sexual arousal, attention, creativity, relaxation, empathy, connectedness, and other cognitive states.

In some embodiments, modulation of neuronal activity underlying multiple sensory domains and/or cognitive states occurs concurrently or in close temporal arrangements.

In some embodiments, the effect of delivering a targeted electrical field to one or more brain regions is a modulation of one or a plurality of the following biophysical or biochemical processes: (i) ion channel activity, (ii) ion transporter activity, (iii) secretion of signaling molecules, (iv) proliferation of the cells, (v) differentiation of the cells, (vi) protein transcription of cells, (vii) protein translation of cells, (viii) protein phosphorylation of the cells, or (ix) protein structures in the cells.

In some embodiments, the TES electrodes are arranged in an array with a shape chosen from: round, elliptical, triangular, square, rectangular, trapezoidal, polygonal, oblong, horseshoe-shaped, hooked, or irregularly-shaped.

In some embodiments, the results of a computational algorithm are used to determine the density, impedance, shape, or other property of TES electrodes, where the shape of a TES electrode is chosen from the group including but not limited to: round, elliptical, triangular, square, rectangular, trapezoidal, polygonal, oblong, horseshoe-shaped, hooked, or irregularly-shaped.

Discomfort from TES stimulation increases with current delivered, so methods and systems that replace a smaller number of electrodes delivering high current with a larger number of electrodes delivering lower currents would be advantageous for improving the comfort of TES. Many embodiments comprises a processor configured to: first, compute an FEM model based on a set of two or more electrodes; and second, determine additional electrode positions and stimulation parameters such that a high current delivered from an anode-cathode pair is replaced by a larger number of electrodes delivering a lower current than the high current while approximately maintaining the induced current in one or more brain regions.

FIG. 9 shows a system diagram comprising electronic system 927 in accordance with many embodiments. The electronic system contains computer hardware 911 comprising power source 912, processor 913, local data storage 914, and network interface 915 configured to communicate via the Internet 917 to remote processor 916 and remote data storage 918. The processor may comprise a processor system, for example.

Optionally, anatomical data derived from tomography from multiple users 901, 902, 903 (and, optionally, additional users as indicated by the ellipses between users 902 and 903) is used to compute Standard Model 908. Processor 913 is configured to compute one or more of signal processing steps 907 in any order and selected from the list including but not limited to: computed tomography of anatomy, image registration, spatial normalization, apply warp-field, segment by tissue type, create tetrahedral mesh, and statistical algorithm to combine subjects' data to form a Standard Model. One or more Standard Model Adjustment Parameter values 906 from user 905 is used to query Standard Model Adjustment database 909. The Standard Model Adjustment database is shown as a component of the local computerized system but in alternative embodiments is contained in a remote database accessible via the Internet. Data from the Standard Model Adjustment database is used to compute Adjusted Standard Model 910 which can be used as the anatomical model input to algorithm to estimate current flow from electrical stimulation 911.

Optionally, user conductivity parameter 904 that relates to the electrical conductivity parameters and the position, shape, size, and composition of array of electrodes 926 is transmitted to the system and, together with pulsing parameter ranges and optimization criteria for pulsed or amplitude modulated TES 920 is used as an input to algorithm to estimate current flow from electrical stimulation 911 in order to estimate the distribution and flow of current in the head and brain. This estimate can be used to define electrode array and stimulation parameter 925.

Optionally, the position, shape, size, and composition of an array of n electrodes 926, targeted brain region 924, and stimulation protocol 923 are transmitted to the system and used together with optimization criterion to reduce maximum or peak current 921 as input to algorithm to estimate current flow from electrical stimulation 911. This estimate can be used to define an array of at least (n+1) electrodes and spatiotemporal stimulation parameters 925 wherein the maximum or peak current delivered from the array is reduced.

Optionally, the modality and other parameters of non-TES form of neuromodulation 922 is transmitted to the system and, together with algorithm to estimate current flow from non-TES neuromodulation 919 is used as an input to algorithm to estimate current flow from electrical stimulation 911 in order to estimate the distribution and flow of current in the head and brain. This estimate can be used to define electrode array and spatiotemporal stimulation parameter 925.

TABLE 1
Conductivity of various tissues and TES components (adapted
from (Dmochowski et al., 2011)) with data on white matter
and grey matter conductivity from (Latikka et al., 2001))

| Tissue | Conductivity (S/m) |
|---|---|
| Grey matter | 0.28 |
| White matter | 0.25 |
| Skull | 0.01 |
| CSF | 1.65 |
| Scalp | 0.465 |
| Muscle | 0.334 |
| Air | 1 × 10⁻¹⁵ |
| Electrode | 5.9 × 10⁷ |
| Gel | 0.3 |

The data of Table 1 show impedance values that can be readily determined by a person of ordinary skill in the art suitable for combination in accordance with embodiments as disclosed herein. The data of the adjustable model comprises a plurality of structures corresponding to a plurality of the tissues shown in Table 1. By adjusting the model as described herein, the locations of the structures of the model can be changed so as to provide improved modeling of the current at the target location. In many embodiments the finite element model comprises a plurality of elements composed of elements corresponding to one or more of the tissues of Table 1, and the locations of these elements are adjusted in response to the subject data as described herein, such that the locations of the adjusted elements correspond to locations of corresponding tissue structures of the user. For example, first elements corresponding to grey matter of the standard model can be moved from locations of the standard model to locations of the subject in response to measurement data of the subject as described herein. One or more additional tissues of Table 1 can be similarly moved with the processor system.
Dmochowski, J. P., Datta, A., Bikson, M., Su, Y., and Parra, L. C. (2011). Optimized multi-electrode stimulation increases focality and intensity at target. Journal of Neural Engineering 8, 046011.
Latikka, J., Kuurne, T., and Eskola, H. (2001). Conductivity of living intracranial tissues. Physics in Medicine and Biology 46, 1611-1616.

While preferred embodiments of the present disclosure have been shown and described herein, it will be obvious to those skilled in the art that such embodiments are provided by way of example only. Numerous variations, changes, and substitutions will be apparent to those skilled in the art without departing from the scope of the present disclosure. It should be understood that various alternatives to the embodiments of the present disclosure described herein may be employed without departing from the scope of the present disclosure. Therefore, the scope of the present invention shall be defined solely by the scope of the appended claims and the equivalents thereof.

What is claimed is:

1. An apparatus for use with a brain of a subject, the apparatus comprising:
an input to receive data of the subject; and
a computer configured with an adjustable model to determine parameters and electrode positions to provide a spatiotemporal pattern of stimulation in order to target one or more regions of the brain with electrical stimulation based on the input.

2. The apparatus as in claim 1, wherein the adjustable model comprises a plurality of structures corresponding to tomography of another subject, and wherein the model is adjusted based on the input in order to align the structures of the model with corresponding structures of the subject.

3. The apparatus of claim 2, wherein each of the plurality of structures corresponds to one or more of grey matter, white matter, skull, cerebrospinal fluid (CSF), scalp, muscle, air, electrode, or gel and wherein a location of each of the plurality of structures is adjusted based on the input.

4. The apparatus as in claim 1, wherein the adjustable model is scaled to the subject in response to the input in order to align structures of the model with structures of the subject.

5. The apparatus of claim 1, wherein the adjustable model comprises a finite element model comprising a mesh composed of a plurality of finite elements, and wherein the mesh and the plurality of finite elements are scaled to the subject based on the input.

6. The apparatus as in claim 1, wherein the computer is configured to decrease a peak current in order to stimulate a target region of the brain based on the input.

7. The apparatus as in claim 1, wherein computer is configured with the adjustable model to estimate a current induced in the brain by transcranial electrical stimulation treatment of the subject, and wherein the computer comprises,
the adjustable model, the adjustable model based at least in part on brain and head anatomy of another subject based on a structural scan of the another subject and stored in a computer readable memory of the computer,
a database or lookup table indicating adjustments to the adjustable model of brain and head anatomy based on at least one adjustment parameter, and
a processor configured to load the adjustable model of brain and head anatomy from the computer readable memory, to determine one or more model adjustments to make in response to querying a database or lookup table, and to compute adjustment to the adjustable model of brain and head anatomy.

8. The apparatus of claim 7, wherein the processor is configured to compute a computational model for estimating current density and direction in the brain based on the input and the adjustable model.

9. The apparatus of claim 7, wherein the processor comprises instructions to determine one or more model adjustment parameters, the one or more model adjustment parameters comprising a subject measurement comprising of one or more of:
a subject's skull, a scalp, a hair, a face, a head, a dura, a brain, a neck, or other part of the body;
a cognitive assessment comprising one or more of a test of motor control, a test of cognitive state, a test of cognitive ability, a sensory processing task, an event related potential assessment, a reaction time task, a motor coordination task, a language assessment, a test of attention, a test of emotional state, a behavioral assessment, an assessment of emotional state, an assessment of obsessive compulsive behavior, a test of social behavior, an assessment of risk-taking behavior, an assessment of addictive behavior, a standardized cognitive task, or a customized cognitive task;
a physiological measurement of the body comprising of one or more of electromyogram (EMG), galvanic skin response (GSR), heart rate, blood pressure, respiration rate, electrocardiogram (EKG), pulse oximetry (e.g. photoplethysmography), heart rate, pupil dilation, eye movement, or gaze direction;
a subject metadatum comprising one or more of gender, height, weight, age, diet, pharmaceutical drugs used, cognitive abilities, cognitive disabilities, or other metadata; or
a subject genetic datum including one or more of microduplication, microdeletion, single nucleotide polymorphism (SNP), aneuploidy, allele, or other genetic data.

10. The apparatus of claim 7, wherein the processor is configured to write the adjusted model of brain and head anatomy to a computer readable memory and determine positions of the electrodes in order to decrease peak current.

11. The apparatus of claim 7, further comprising a communication system for transmitting information between a remote processor and a transcranial electrical stimulation system controller.

12. The apparatus of claim 11, wherein the communication system comprises the Internet.

13. The apparatus of claim 11, wherein the transmitted information comprise a Model Adjustment Parameter transmitted from a transcranial electrical stimulation system controller to a remote server.

14. The apparatus of claim 11, wherein the transmitted information comprises a transcranial electrical stimulation electrode montage transmitted to a transcranial electrical stimulation system controller.

15. The apparatus of claim 11, wherein the transmitted information comprises a transcranial electrical stimulation electrostimulation protocol transmitted to a TES system controller.

16. The apparatus of claim 11, wherein the transmitted information comprises a transcranial electrical stimulation system parameters selected from the group consisting of: firmware version, number of electrodes, location of electrodes, size and shape of electrodes, stimulation protocol history, capacity of the system to deliver direct current stimulation and/or alternating current stimulation, battery charge remaining, maximum current deliverable, constraints on anode-cathode pairs that can be created from available electrodes, and other information about a TES system.

17. The apparatus of claim 11, wherein the transmitted information comprises at least one brain target for transcranial electrical stimulation.

18. The apparatus of claim 11, wherein the transmitted information relates to the outcome of one or more previous TES sessions, where the transmitted data comprises one or more of a subjective assessment by the subject or another individual, a cognitive assessment, a brain recording or other physiological measurement, or other outcome assessment.

19. The apparatus of claim 11, wherein the transmitted information comprises instructions to a TES system controller to adjust one or more parameters comprising one or more of an electrode position, anode-cathode pairing of two or more electrodes, current delivered from an anode-cathode pair of electrodes, timing of stimulation from electrodes, or frequency of alternating current stimulation, or other TES parameter.

20. An apparatus for determining a transcranial electrical stimulation electrode montage and electrostimulation protocol, the apparatus comprising:
a processor configured to compute an FEM model based on a set of two or more electrodes and determine additional electrode positions and stimulation parameters.

21. The apparatus of claim 20, wherein the processor comprises instructions such that a high current delivered from an anode-cathode pair is replaced by a larger number of electrodes delivering a lower current than the high current while approximately maintaining the induced current in one or more brain regions.

22. The apparatus of claim 1, further comprising components to deliver another brain stimulation by one or more of transcranial ultrasound neuromodulation, transcranial magnetic stimulation (TMS), deep brain stimulation (DBS), optogenetic stimulation, one electrode or an array of electrodes implanted on the surface of the brain or dura (electrocorticography (ECoG) arrays), or radio-frequency stimulation.

23. The system of claim 22, wherein the components to deliver another brain stimulation are triggered with a pre-defined temporal relationship relative to a TES protocol.

24. The system of claim 22, wherein the components to deliver another brain stimulation is delivered concurrently with a TES protocol.

25. The apparatus of claim 1, further comprising circuitry and multiple electrode pairs configured to be pulsed at defined latencies relative to each other to target electrical stimulation to one or more brain regions.

26. The apparatus of claim 25, wherein the circuitry and multiple electrodes are configured for pulses comprising one or more of direct current stimulation, alternating current stimulation, or both direct current stimulation or alternating current stimulation.

27. The apparatus of claim 25, wherein the circuitry and multiple electrode pairs are configured for pulsed electrical stimulation from with a phase shift between pulses from different sets of electrodes of less than 10 ms.

28. A method of treating a patient, the method comprising:
receiving input data of the subject; and
adjusting a model to determine parameters and electrode positions to provide a spatiotemporal pattern of stimulation in order to target one or more regions of the brain with electrical stimulation based on the input.
